(19) United States
(12) Patent Application Publication
(10) Pub. No.: US 2006/0051437 A1
(43) Pub. Date: Mar. 9, 2006

(54) Process for preparing a ginger fraction and the use thereof for inhibiting human CYP enzymes

(75) Inventors: Thomas Ebner, Ummendorf (DE); Eva Ludwig-Schwellinger, Ummendorf (DE); Stefan Matthias Blech, Warthausen (DE)

Correspondence Address: EDELL, SHAPIRO & FINNAN, LLC, 1901 RESEARCH BOULEVARD, SUITE 400, ROCKVILLE, MD, 20850, US

(73) Assignee: Boehringer Ingelheim International GmbH, Ingelheim (DE)

(21) Appl. No.: 11/209,322

(22) Filed: Aug. 23, 2005

(30) Foreign Application Priority Data
Aug. 28, 2004 (DE) 10 2004 041 716

(51) Int. Cl. A61K 36/9068

(52) U.S. Cl. 424/756

(57) ABSTRACT

The present invention relates to a process for preparing a ginger fraction, the fraction prepared by this process and the use thereof on its own or combined with drugs for inhibiting human cytochrome P450 (CYP) enzymes (particularly cytochrome P450 3A4, CYP3A4) for positively influencing the oral bioavailability and pharmacokinetics of active substances.

The present invention relates to a process for preparing a ginger fraction, the fraction prepared by this process and the use thereof on its own or combined with drugs for inhibiting human cytochrome P450 (CYP) enzymes (particularly cytochrome P450 3A4, CYP3A4) for positively influencing the oral bioavailability and pharmacokinetics of active substances.

### BACKGROUND TO THE INVENTION

Cytochrome P450 (CYP) enzymes play a central part in drug metabolism. They are found primarily in the liver but also in the intestinal wall, lungs, kidneys and other extrahepatic organs. Orally administered active substances may demonstrate poor bioavailability as a result of the so-called “first-pass effect”, for example those active substances which are subject to metabolisation in the intestinal wall or liver before reaching the systemic circulation.

If the first-pass metabolism is inhibited, a significant increase in the bioavailability of orally administered active substances can be achieved (Gibbs, Megan A. and Hosea, Natalie A.: Factors affecting the clinical development of cytochrome P450 3A substrates; Clin. Pharmacokinet. 2003; 42(11), 969-984). Many examples of active substance-active substance interactions which result in a bioavailability higher than that of the active substance administered are based on such effects. In such cases the first-pass metabolism of the active substance is inhibited by another active substance administered simultaneously.

Inhibiting the first-pass metabolism may, in addition to increasing the bioavailability of an active substance, significantly reduce the variability in bioavailability, which is known to increase as absolute bioavailability decreases. By reducing the variability in bioavailability the therapeutic success of an oral drug therapy is critically improved, as there is a lower incidence of exposure to excessively high drug levels (risk of unwanted side effects) or excessively low drug levels (risk of therapeutic failure). Such effects may have certain advantages in drug therapy. For example the HIV drug lopinavir has inadequate bioavailability because of the first-pass metabolism by CYP3A4. If it is administered in a fixed-dose combination with ritonavir, which is a potent inhibitor of CYP3A4, a significantly higher oral bioavailability is achieved for lopinavir.

However, there are only limited possibilities of combining an active substance with poor oral bioavailability with another active substance or substance resembling an active substance in order to reduce the first-pass effect. This is due mainly to the mode of activity of the additional active substance. Thus, for example, too low a bioavailability of a cardiovascular drug cannot be increased by simultaneously giving an anti-retroviral active substance (indicated for HIV infection) to non-HIV-infected patients for ethical reasons. Even permitted active substances are not licensed for the purpose of inhibiting enzymes that metabolise active substances.

Instead, drug additives may be useful in this respect. For example, some constituents of grapefruit juice are potent inhibitors of CYP3A4 and drug transporters in the intestinal wall. The prior art contains numerous examples demonstrating that taking the drug together with grapefruit juice has dramatic effects on the pharmacokinetics, safety and efficacy of orally administered active substances, such as e.g. simvastatin, cyclosporin A, terfenadine etc. (Ameer, Barbara and Weintraub, Randy A.: Drug interactions with grapefruit juice; Clin. Pharmacokinet. 1997; 33(2):103-121).

### DESCRIPTION OF THE INVENTION

Ginger (Zingiber officinalis) is a traditional food ingredient in many parts of the world and is also used as a phytopharmaceutical for various applications. For example, powdered ginger root is available as a preparation for preventing seasickness. It contains about 5 to 8% of a viscous liquid balsam (oleoresin), which contains a non-steam-volatile peppery or hot fraction as well as a volatile ethereal oil fraction. The pale yellow ethereal oil makes up about 20 to 25% of the oleoresin. The composition of the ethereal oil is subject to considerable fluctuations depending on its origin. It contains as its main ingredient sesquiterpene hydrocarbons of the bisabolone type, particularly (−)-α-zingiberene and also (−)-β-sesquiphellandrene, (−)-β-bisabolene, (+)-ar-curcumene and acyclic α-farnesene (Deutsche Apothekerzeitung 1997, 137(47), 40-46).

The main component of the hot fraction, making up about 25% of the oleoresin, constitutes the homologous series of the gingerols (HagerROM 2002: Zingiberis rhizoma, Springer Verlag, Heidelberg).

Surprisingly, in vitro tests on the inhibition of CYP by various active substances and other compounds have shown that potent inhibition of various human CYP's may be achieved by means of a ginger fraction obtained by an extraction process according to the invention.

This fraction shows a higher inhibitory potency (IC₅₀ in the range below 1 μg/ml) both compared to the commercially available total ginger extract (the so-called oleoresin) and also compared to the highly volatile fraction of ethereal ginger oil (IC₅₀ approx. 23 μg/ml), which is separated off in the first extraction step.

The fraction obtained here is poorly soluble in hexane and differs in this characteristic from the fraction of the ethereal oil, which has already been shown to inhibit CYP3A4 (U.S. Pat. No. 5,665,386).

The process according to the invention starts from a commercially obtainable oleoresin and comprises a number of extraction steps using organic and aqueous solvents.

A first object of the present invention is thus a process for isolating a ginger fraction while separating off the ethereal oil, comprising the steps of
(a) extracting an oleoresin with a non-polar organic solvent;
(b) extracting the combined residues from step (a) with warm water and discarding the supernatant.

The residues thus obtained have an IC₅₀ value of 0.9 μg/mL for CYP3A4. This value is achieved with human liver microsomes in the experiment described in the experimental section.

In a preferred embodiment the residue thus obtained is further purified by a process comprising the steps of
(c) extracting the combined residues from step (b) with warm alcohol and
(d) concentrating the combined supernatants from step (c).

The fraction obtained in step (d) may be dissolved in an alcohol, preferably methanol or ethanol, and optionally further fractionated, for example by solid phase extraction and stepwise elution.

Non-polar organic solvents which may be used in step (a) include according to the invention low-boiling alkane solvents such as, for example, hexane, heptane, octane, pentane or cyclohexane, petrochemical distillates, propellants and solvents such as for example petrol, kerosene, petroleum ether, petroleum and other low-boiling, volatile and non-polar solvents such as for example diethyl ether, tert.-butyl-methylether, tetrahydrofuran, benzene, toluene and xylenes, while hexane is preferably used.

The alcohol used in steps (c) and (e) may be selected from among methanol, ethanol, isopropanol, n-propanol, n-butanol and other positionally isomeric butanols, n-pentanol and other positionally isomeric pentanols and may be identical or different. Preferably, methanol is used. The extraction agent in each case is used in amounts of from 4 to 10 mL/g, preferably 4 to 7 mL/g, of the oleoresin used.

The aqueous extractions are preferably carried out at a temperature of from 50 to 80° C., particularly preferably 65 to 75° C.

As an alternative to this method extractions may also be carried out with suitable aqueous organic acids or, instead of liquid-liquid extraction with organic solvents, solid phase extractions with suitable non-polar absorbents may also be carried out.

The extractions carried out in steps (a), (b) and (c) may be carried out once or several times, and the phases containing the desired product from the various extractions of one step may be combined. Preferably the extraction is carried out three times in each step and the phases containing the product are combined. The combined phases are then further processed.

A second object of the present invention is the ginger fraction according to the invention, which may be obtained by one of the processes according to the invention.

A ginger fraction which contains at least one compound of general formulae 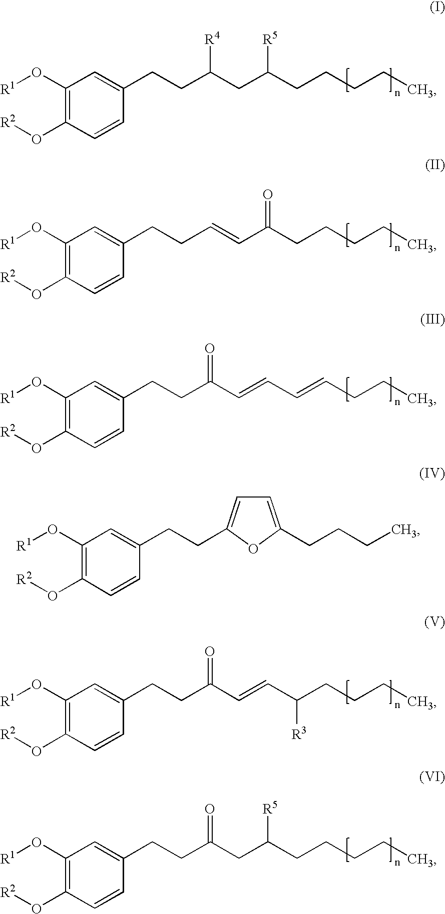
wherein
n denotes the number 1, 2 or 3,
R¹ denotes H, CH₃,
R² denotes H, CH₃,
R³ denotes H, OH, OCH₃,
R⁴ denotes H, O, OH, OCH₃, OC(O)CH₃ and
R⁵ denotes H, O, OH, OCH₃, OC(O)CH₃,
   one of the enantiomers or diastereomers thereof, is preferred.

The following are mentioned as particularly preferred compounds of the above general formulae I to VI: 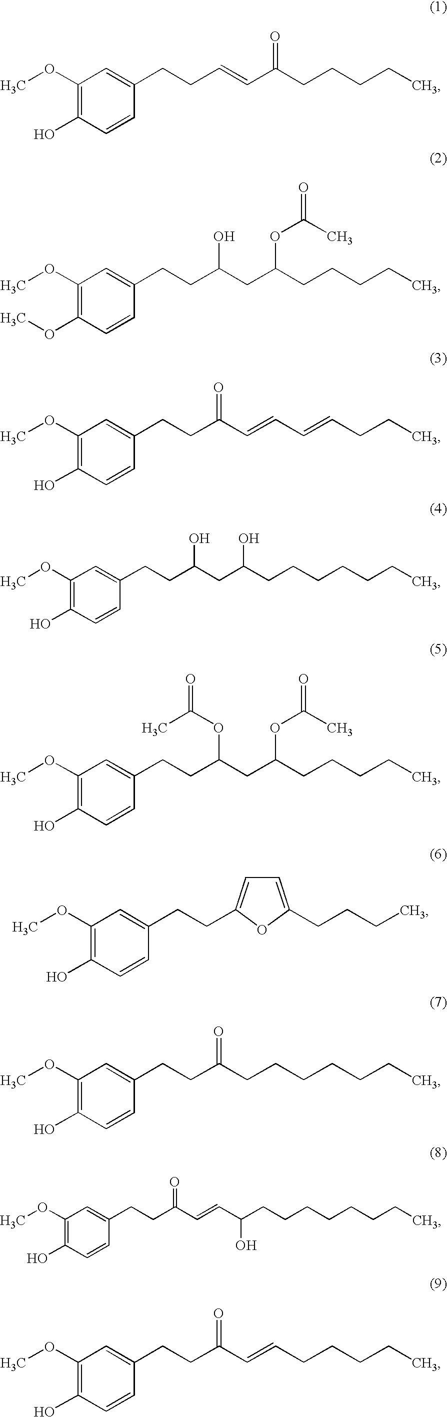 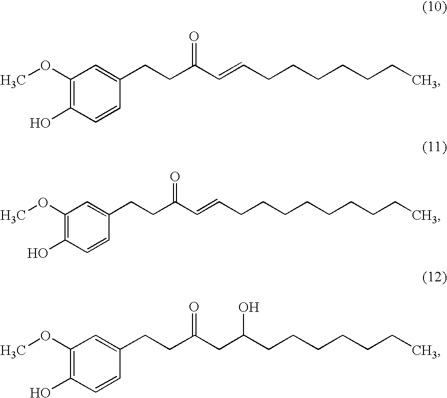
the enantiomers and the diastereomers thereof.

The compounds of general formulae I to VI were identified from the ginger fraction obtained according to the invention. In order to characterise this ginger fraction more precisely and establish its contents, it was suitably further purified with the aim of isolating purified fractions of individual ingredients.

In order to do this, the ginger fraction obtained according to the invention was further purified by solid phase extraction on a C18 phase. The eluant of the solid phase extraction was dried out and investigated further by semipreparative high pressure liquid chromatography (HPLC). This was done by injecting fairly small aliquots of 5 to 10 mg into the semipreparative HPLC system. The eluant of the HPLC column was then collected in 60 to 65 individual fractions and each of the fractions thus obtained was investigated for its inhibitory effect on various P450 test reactions. The results of these investigations showed clearly defined zones (peaks) of higher inhibitory potency.

To clarify the chemical structure of the constituents of the individual fractions, selected samples were further purified and concentrated by repeated HPLC and then investigated by mass spectrometry and NMR spectroscopy.

Compounds (1) to (12) identified according to the invention are the typical ingredients of the non-volatile hot fraction of ginger which have already been sufficiently described in the literature. In addition to various modification products of gingerol and the various homologues thereof, a known main ingredient of ginger, [8]-gingerol (12), and also [6]-shogaol (9), the breakdown product of the main ingredient [6]-gingerol which is present in the largest amount, were also found.

This confirmed that the ginger fraction prepared by the process described is derived from the non-volatile hot fraction and the inhibition of the CYP enzymes is brought about by ingredients of the gingerol type and the structural modifications and breakdown products thereof.

A third object of the present invention is the use of the ginger fraction according to the invention and one or more of the compounds of general formulae (I) to (VI) isolated therefrom for preparing a pharmaceutical composition for inhibiting cytochrome P450 enzymes, particularly cytochrome P450 3A4, 1A2, 2C19 and 2C9. Preferably, also, the cytochromes P450 1A2, P450 2C19 and P450 2C9 are inhibited.

A fourth object of the present invention is the use of one or more compounds of general formulae 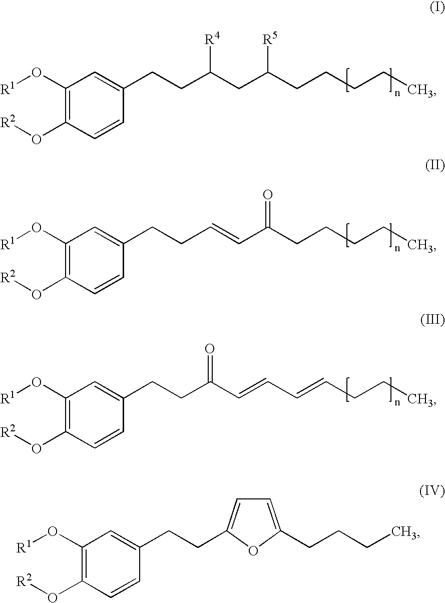
wherein
n denotes the number 1, 2 or 3,
R¹ denotes H, CH₃,
R² denotes H, CH₃,
R³ denotes H, OH, OCH₃,
R⁴ denotes H, O, OH, OCH₃, OC(O)CH₃ and
R⁵ denotes H, O, OH, OCH₃, OC(O)CH₃,
   the enantiomers or diastereomers thereof, for preparing a pharmaceutical composition for inhibiting cytochrome P450 1A2, 2C9 and 2C19.

The following compounds of general formulae I to IV are mentioned as being particularly preferred: 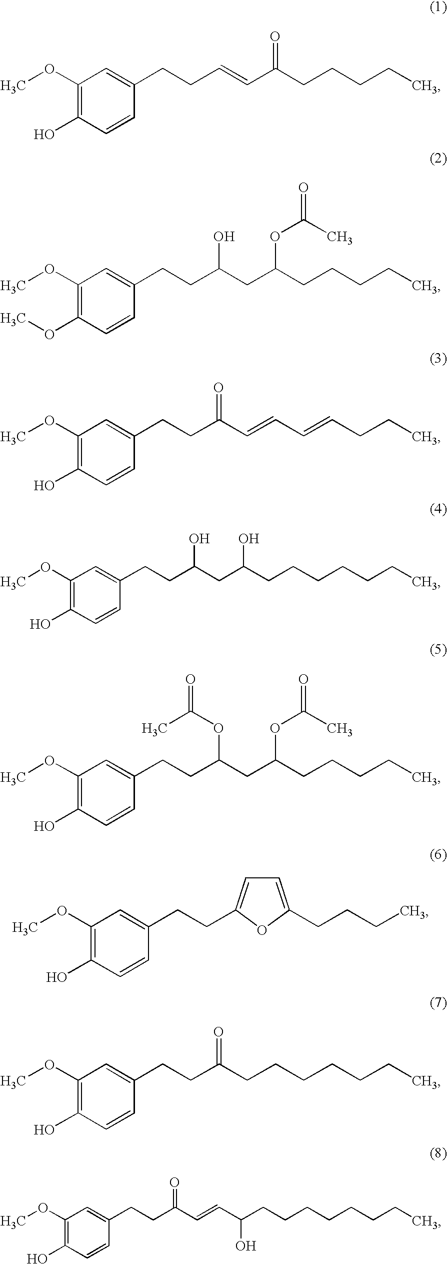
and the enantiomers and the diastereomers thereof.

A fifth object of the present invention is the use of the ginger fraction according to the invention as well as one or more of the compounds of general formulae (I) to (VI) isolated therefrom, in conjunction with a pharmaceutical composition for preparing a pharmaceutical composition for inhibiting human cytochrome P450 (CYP) enzymes, particularly cytochrome P450 3A4, 1A2, 2C19 and 2C9, for positively influencing the oral bioavailability and pharmacokinetics of active substances.

Preferably also, the cytochromes P450 1A2, 2C19 and 2C9 are inhibited.

Many active substances have low oral bioavailability, caused by the so-called first-pass metabolism. This is the metabolic breakdown of orally administered active substances in the small intestine or in the liver, even before they are able to travel through the bloodstream to their target organ.

The active substances mentioned previously, i.e. the pharmacologically active constituents of drugs, may be selected from among the drugs for acting upon the cardiovascular system in its widest sense, including those substances which serve to influence the composition of the blood (e.g. blood lipids); drugs acting on the central nervous system; drugs for treating metabolic disorders (e.g. diabetes mellitus); drugs for treating inflammatory processes in the widest sense; drugs for influencing the immune system; drugs for treating infections by bacteria, protozoa, multi-cellular parasites, viruses, fungi or prions; drugs for stopping or alleviating degenerative processes in various organs, particularly the brain, and drugs for treating cancer.

By the term “drugs” are meant substances and preparations of substances which are intended, by administration to or in the human or animal body,
1. to cure, alleviate, prevent or detect diseases, ailments, physical injury or pathological disorders;
2. to show up the nature, state or functions of the body or mental states;
3. to replace active substances or bodily fluids produced by the human or animal body;
4. to ward off, eliminate or render harmless pathogens, parasites or substances alien to the body or
5. to influence the nature, state or functions of the body or mental states.

Cytochrome P450 (CYP) enzymes in this case are enzymes from the family of the cytochrome P450 monooxygenases which are involved in the metabolism of drugs according to current scientific knowledge. In particular they are all P450 enzymes of the families CYPLA, CYP1B, CYP2A, CYP2B, CYP2C, CYP2D, CYP2E, CYP2F, CYP2J, CYP3A, CYP4A.

A sixth object of the present invention relates to a process for preparing a pharmaceutical composition for increasing the bioavailability of a pharmaceutical compound for oral administration, comprising orally administering the pharmaceutical compound together with a ginger fraction according to one of claims 8 to 10 to a person requiring such treatment, the ginger fraction being administered in an amount which is needed to increase the bioavailability of the pharmaceutical compound as compared with administration of the pharmaceutical compound on its own. The pharmaceutical compound is characterised in that it is metabolised by cytochrome P450 enzymes, preferably by P450 3A4, 1A2, 2C9 and 2C19.

A seventh object of the present invention relates to a pharmaceutical formulation containing the ginger fraction which may be obtained according to the invention or at least one compound of general formulae I to VI, the enantiomers or diastereomers thereof, optionally together with one or more pharmaceutically acceptable carriers and/or diluents for improving the oral bioavailability and pharmacokinetics of active substances.

Preferably a pharmaceutical composition of this kind consists of two or more components which are optionally physically separate from one another and comprises:
(a) a first component consisting of the ginger fraction according to the invention and one or more pharmaceutically acceptable diluents and/or carriers; and
(b) a second component containing a pharmaceutical composition, comprising a pharmaceutical compound which is metabolised by cytochrome P450 enzymes, and one or more pharmaceutically acceptable diluents and/or carriers.

In a preferred pharmaceutical composition the first component consists of at least one compound of general formulae I to VI, the enantiomers or diastereomers thereof.

In a more preferred pharmaceutical composition the first component consists of at least one compound of formulae (1) to (12), the enantiomers or diastereomers thereof.

The pharmaceutical composition contained in the second component is preferably metabolised by the enzymes cytochrome P450 1A2, 3A4, 2C9 and 2C19.

As pharmaceutically acceptable carriers and/or diluents, maize starch, lactose, glucose, microcrystalline cellulose, magnesium stearate, polyvinylpyrrolidone, citric acid, tartaric acid, water, water/ethanol, water/glycerol, water/sorbitol, water/polyethyleneglycol, propyleneglycol, cetylstearylalcohol, carboxymethylcellulose or fatty substances such as hard fat or suitable mixtures thereof may be incorporated in the usual way into conventional galenic preparations such as tablets, coated tablets, capsules, powders, suspensions, solutions, metered dose aerosols or suppositories.

Experimental Section

10 g of an oleoresin (Eramex Aromatics GmbH) are extracted three times with 50 mL hexane and the supernatant (organic phase) is discarded. The residues are combined and extracted three times with 40 mL of water heated to 70° C. The supernatant is discarded again and the combined residues are extracted three times with 40 mL of methanol heated to 70° C. The residue is discarded The supernatant obtained is concentrated by rotary evaporation and dissolved in methanol again. Diagram 1 shows an overview of the extraction process of the ginger fraction according to the invention of the oleoresin with separation of the ethereal oils.

The ginger fraction thus obtained was then purified further by solid phase extraction on a C18 phase. The eluant of the solid phase extraction was dried and investigated further by semipreparative high pressure liquid chromatography (HPLC). For this, small aliquots of 5 to 10 mg were injected into the semipreparative HPLC system. The eluant of the HPLC column was then collected in 60 to 65 individual fractions and each of the fractions thus obtained was investigated for its inhibitory effect on various P450 test reactions. The results of these investigations (FIGS. 1 to 4) showed clearly demarcated zones (peaks) of higher inhibitory potency.

Experiments with Human Liver Microsomes

Test (A):

Erythromycin N-demethylation is Used as a Specific Test Reaction for CYP 3A4

The ginger fraction or the various fractions of the extraction process are investigated for their inhibition of CYP. This involves incubating 100 μg of human liver microsomes with 0.01 to 100 μg of ginger fraction and 7.34 μg (10 nmol) of erythromycin at pH 7.4 in the presence of NADPH. The inhibition of the CYP activity is determined by comparison with control incubations with no ginger extract (with the same concentration of solvent).

Test B:

Phenacetin O-deethylation is Used as a Specific Test Reaction for CYP 1A2.

The ginger fraction or the various fractions of the extraction process are investigated for their inhibition of CYP. This involves incubating 125 mg of human liver microsomes with 0.01 to 100 mg ginger fraction and 5 nmol phenacetin at pH 7.4 in the presence of NADPH (1 mM) in a total volume of 250 μl. The inhibition of the CYP activity is determined by comparison with control incubations with no ginger extract (with the same concentration of solvent).

Test C:

S-Mephenytoin 4′-hydroxylation is Used as a Specific Test Reaction for CYP 2C19.

The ginger fraction or the various fractions of the extraction process are investigated for their inhibition of CYP. This involves incubating 125 mg of human liver microsomes with 0.01 to 100 mg ginger fraction and 12.5 nmol S-mephenytoin at pH 7.4 in the presence of NADPH (1 mM) in a total volume of 250 μl. The inhibition of the CYP activity is determined by comparison with control incubations with no ginger extract (with the same concentration of solvent).

Test D:

Tolbutamide Hydroxylation is Used as a Specific Test Reaction for CYP 2C9.

The ginger fraction or the various fractions of the extraction process are investigated for their inhibition of CYP. This involves incubating 125 mg of human liver microsomes with 0.01 to 100 mg ginger fraction and 37.5 nmol tolbutamide at pH 7.4 in the presence of NADPH (1 mM) in a total volume of 250 μl. The inhibition of the CYP activity is determined by comparison with control incubations with no ginger extract (with the same concentration of solvent).

### BRIEF DESCRIPTION OF THE FIGURES

Diagram 1 shows an overview of the process for extracting the ginger fraction according to the invention of an oleoresin while separating off the ethereal oils.

FIG. 1 shows the HPLC separation of a ginger extract and the measurement of the inhibitory potency relative to CYP3A4 for the eluted HPLC fractions (collecting period: 1 minute).

FIG. 2 shows the HPLC separation of a ginger extract and the measurement of the inhibitory potency relative to CYP1A2 for the eluted HPLC fractions (collecting period: 1 minute).

FIG. 3 shows the HPLC separation of a ginger extract and the measurement of the inhibitory potency relative to CYP2C19 for the eluted HPLC fractions (collecting period: 1 minute).

FIG. 4 shows the HPLC separation of a ginger extract and the measurement of the inhibitory potency relative to CYP2C9 for the eluted HPLC fractions (collecting period: 1 minute).

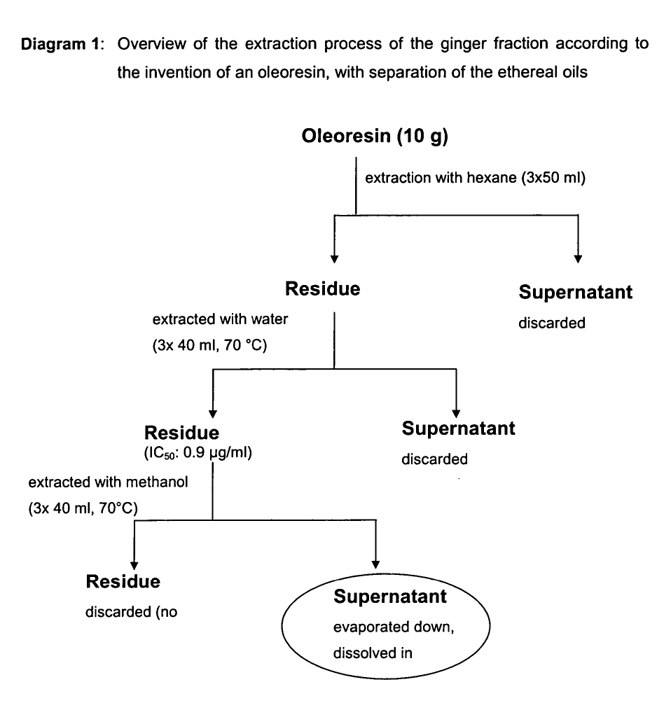

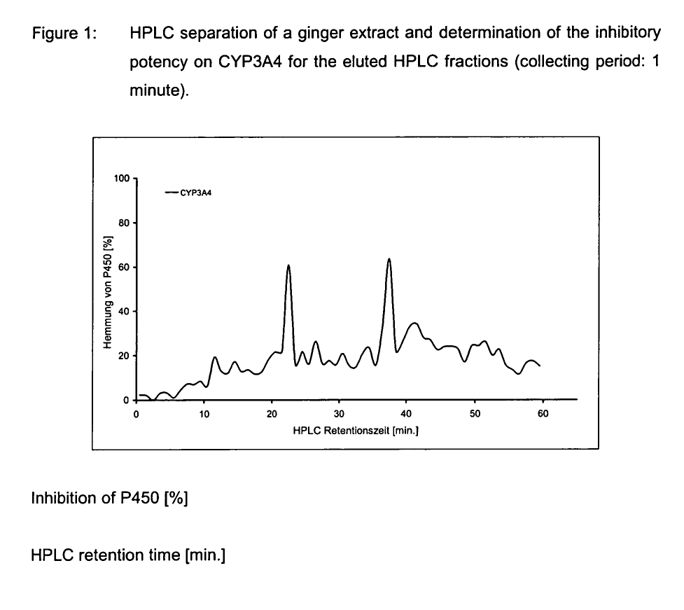

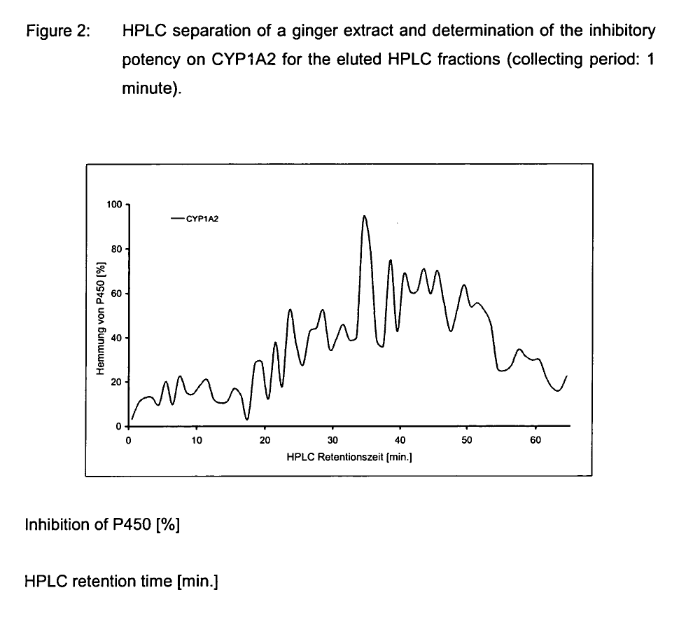

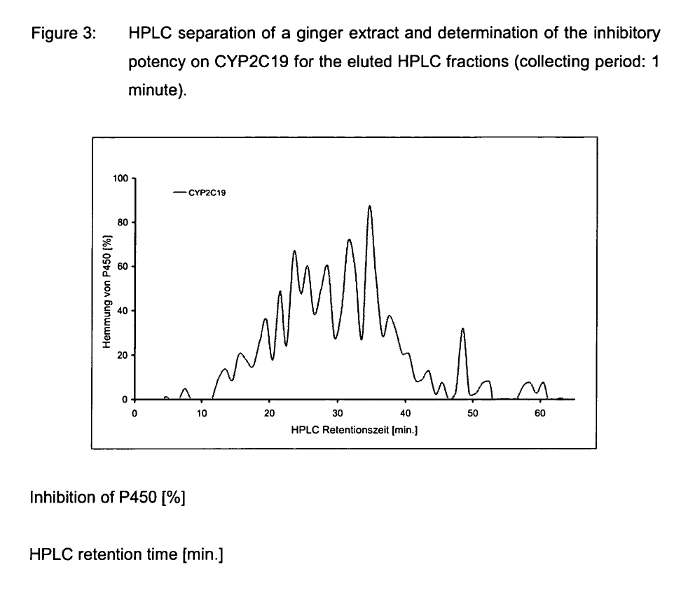

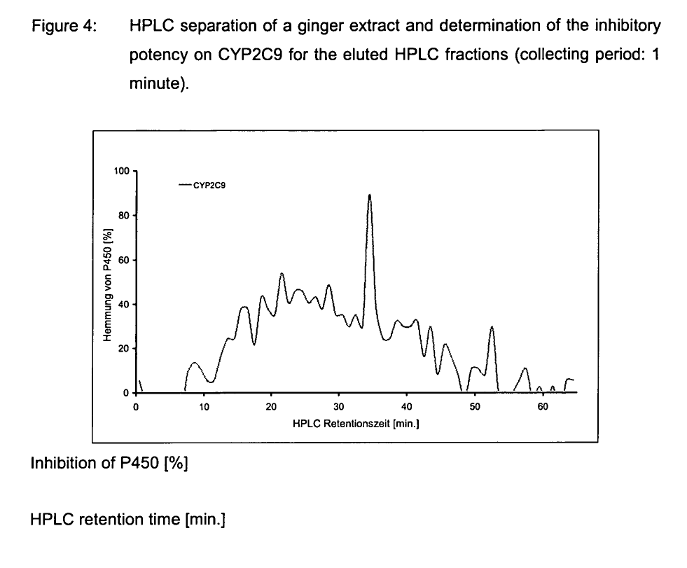

1. Process for isolating a ginger fraction while separating off the ethereal oil, comprising the steps of
(a) extracting an oleoresin with a non-polar organic solvent;
(b) extracting the combined residues from step (a) with warm water and discarding the supernatant.

2. Process according to claim 1, characterised in that the combined residues obtained in step (b) are further purified by a process comprising the steps of (c) extracting with warm alcohol and (d) concentrating the combined supernatants from step (c).

3. Process according to claim 1, characterised in that in step (a) a low-boiling alkane solvent, a petrochemical distillate, a propellant or another low-boiling, volatile and non-polar solvent is used as non-polar organic solvent.

4. Process according to claim 1, characterised in that in step (a) hexane is used as non-polar organic solvent.

5. Process according to claim 2, characterised in that in step (c) methanol, ethanol, isopropanol, n-propanol, n-butanol or another positionally isomeric butanol, n-pentanol or another positionally isomeric pentanol is used as alcohol.

6. Process according to claim 2, characterised in that in step (c) methanol is used as alcohol.

7. Process according to claim 2, characterised in that the extraction agent used in steps (a), (b) and (c) is used in each case in amounts of 4 to 10 mL/g of the oleoresin used.

8. Ginger fraction obtained from the process according to claim 1.

9. Ginger fraction according to claim 8, characterised in that 4 the ginger fraction contains at least one compound of the following general formulas: 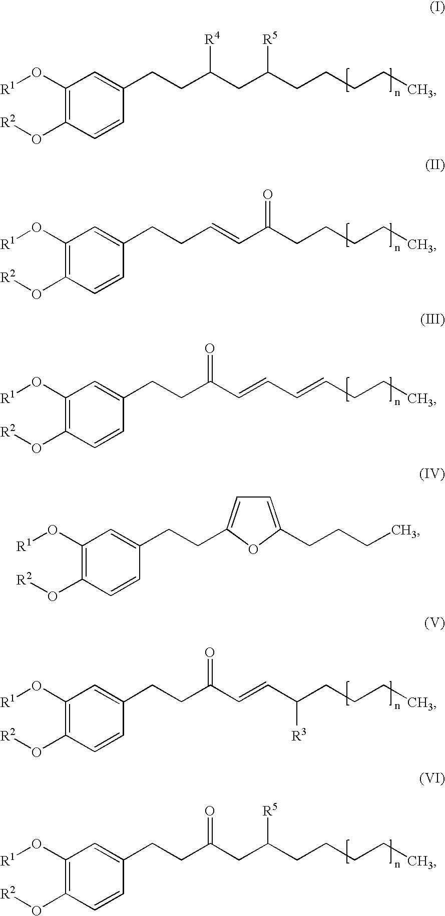
wherein
n denotes the number 1, 2 or 3,
R¹ denotes one of H and CH₃,
R² denotes one of H and CH₃,
R³ denotes one of H, OH and OCH₃,
R⁴ denotes one of H, O, OH, OCH₃ and OC(O)CH₃ and
R⁵ denotes H, O, OH, OCH₃ and OC(O)CH₃, and
one of the enantiomers or diastereomers thereof.

10. Ginger fraction according to claim 8, characterised in that the ginger fraction contains at least one of the following compounds: 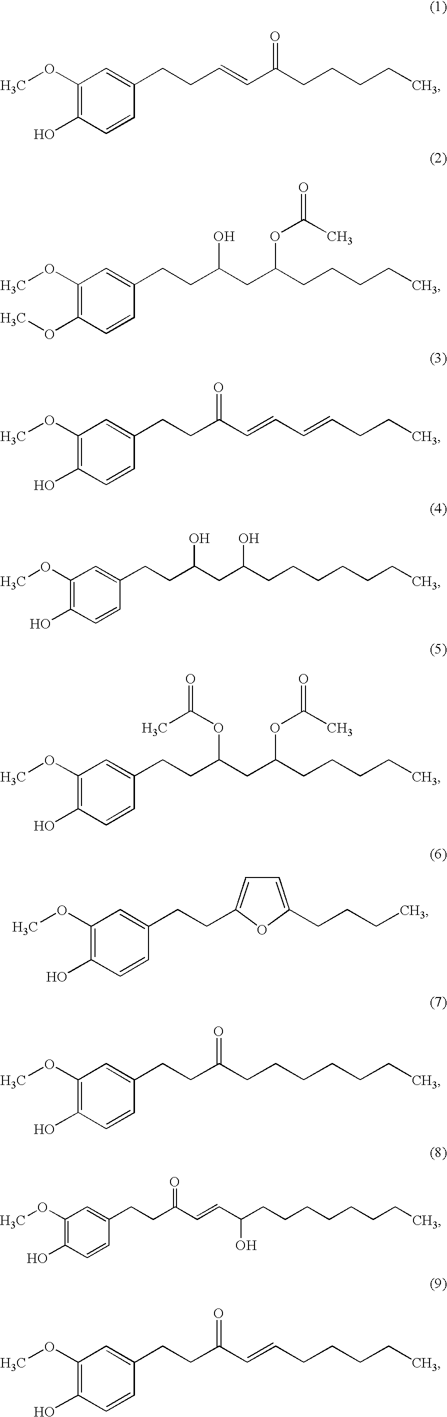 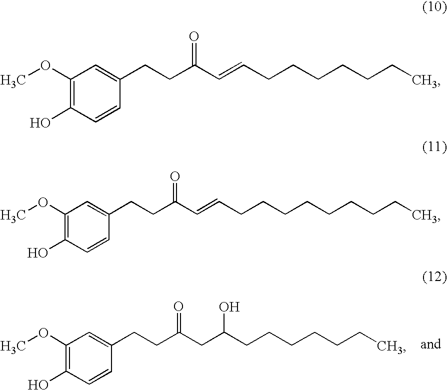
the enantiomers and the diastereomers thereof.

11. A pharmaceutical composition for inhibiting effective to inhibit cytochrome P450 enzymes, wherein the pharmaceutical composition comprises a ginger fraction according to claim 8.

12. A pharmaceutical composition effective to inhibit human cytochrome P450 enzymes, wherein the pharmaceutical composition comprises at least one compound according to claim 9.

13. The pharmaceutical composition of claim 11, wherein the pharmaceutical composition inhibits the cytochromes P450 3A4, 1A2, 2C19 and 2C9.

14. The pharmaceutical composition of claim 11, wherein the pharmaceutical composition inhibits the cytochromes P450 1A2, 2C9 and 2C19.

15. A pharmaceutical composition effective to inhibit cytochrome P450 1A2, 2C9 and 2C19, wherein the pharmaceutical composition comprises one or more compounds having at least one of the general formulas: 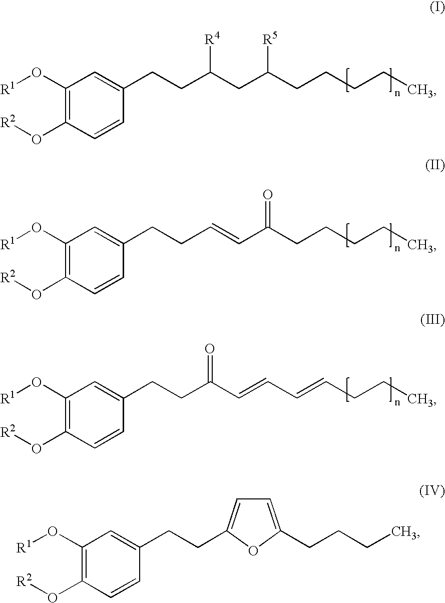
wherein
n denotes the number 1, 2 or 3,
R¹ denotes one of H and CH₃,
R² denotes one of H and CH₃,
R⁴ denotes one of H, O, OH, OCH₃ and OC(O)CH₃ and
R⁵ denotes H, O, OH, OCH₃ and OC(O)CH₃, and
the enantiomers or diastereomers thereof.

16. The pharmaceutical composition according to claim 15, characterised in that the compounds of general formulas I to IV are selected from: 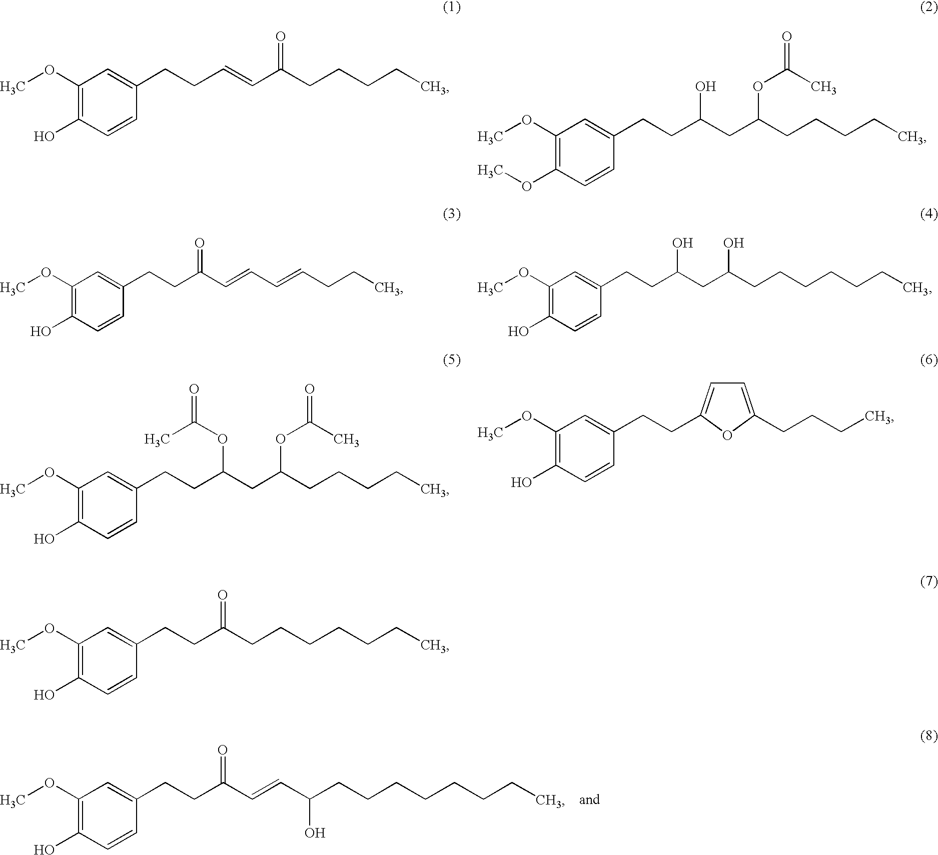
the enantiomers and the diastereomers thereof.

17. A pharmaceutical composition effective to inhibit human cytochrome P450 (CYP) enzymes, wherein the pharmaceutical composition comprises the ginger fraction according to claim 8.

18. A pharmaceutical composition effective to inhibit human cytochrome P450 (CYP) enzymes wherein the pharmaceutical composition comprises one or more compounds according to claim 9.

19. The pharmaceutical composition of claim 17, characterised in that the enzyme is cytochrome P450 3A4, 1A2, 2C19 or P450 2C9.

20. The pharmaceutical composition of claim 17, characterised in that the enzyme is cytochrome P450 1 A2, 2C19 or P450 2C9.

21. Process for increasing the bioavailability of a pharmaceutical compound to be administered orally, comprising oral administration of the pharmaceutical compound together with a ginger fraction according to claim 8 to a person requiring treatment with the pharmaceutical compound, the ginger fraction being administered in an amount which is necessary in order to increase the bioavailability of the pharmaceutical compound as compared with administering the pharmaceutical compound without the ginger fraction.

22. Process according to claim 21, characterised in that the pharmaceutical compound is metabolised by cytochrome P450 3A4 enzymes.

23. Process according to claim 21, characterised in that the pharmaceutical compound is metabolised by cytochrome P450 1A2 enzymes.

24. Process according to claim 21, characterised in that the pharmaceutical compound is metabolised by cytochrome P450 2C9 enzymes.

25. Process according to claim 21, characterised in that the pharmaceutical compound is metabolised by cytochrome P450 2C19 enzymes.

26. Pharmaceutical formulation comprising one or more compounds having at least one of the general formulas: 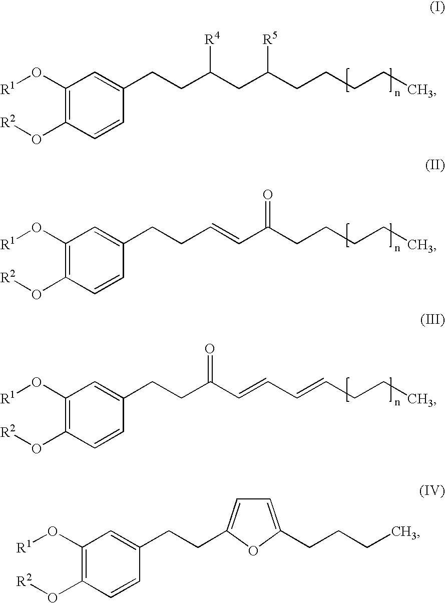
wherein
n denotes the number 1, 2 or 3,
R¹ denotes one of H and CH₃,
R² denotes one of H and CH₃,
R⁴ denotes one of H, O, OH, OCH₃ and OC(O)CH₃, and
R⁵ denotes H, O, OH, OCH₃ and OC(O)CH₃, and
the enantiomers or diastereomers thereof;
wherein the pharmaceutical formulation further comprises one or more inert carriers and/or diluents.

27. Pharmaceutical formulation comprising one or more compounds having at least one of the following formulas: 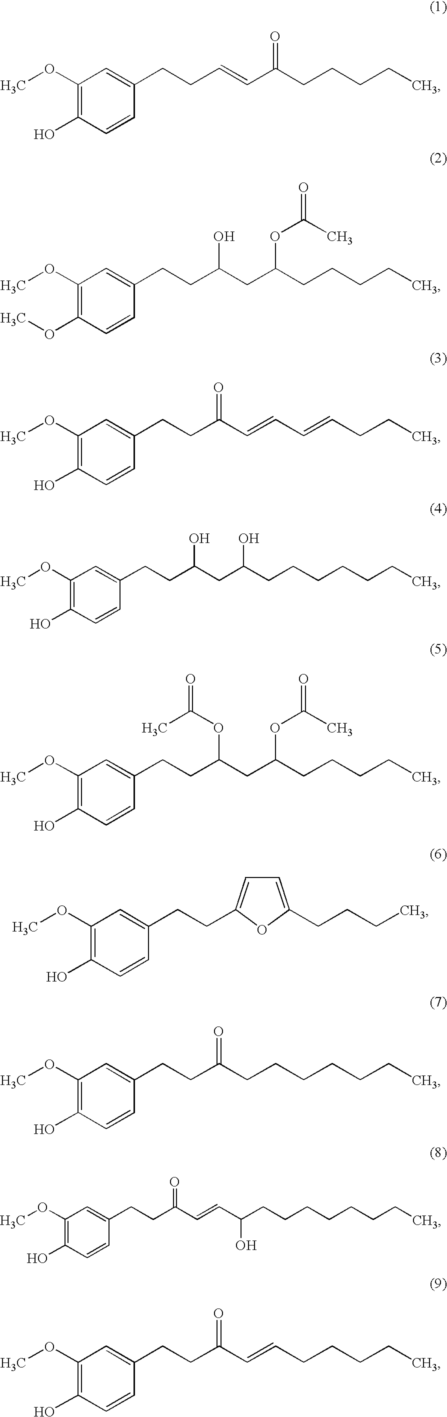 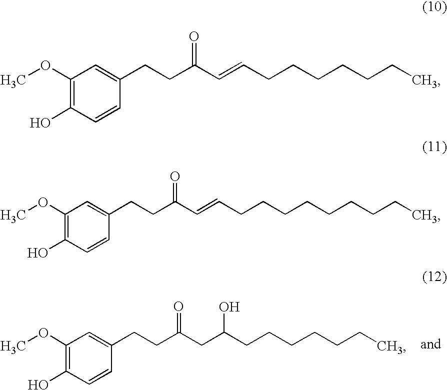
the enantiomers and the diastereomers thereof;
wherein the pharmaceutical formulation further comprises one or more inert carriers and/or diluents.

28. Pharmaceutical composition consisting of two or more components which are physically separate from one another, comprising:
(a) a first component consisting of the ginger fraction according to claim 8 and one or more pharmaceutically acceptable diluents and/or carriers; and
(b) a second component containing a pharmaceutical composition, comprising a pharmaceutical compound which is metabolised by cytochrome P450 enzymes, and one or more pharmaceutically acceptable diluents and/or carriers.

29. Pharmaceutical composition according to claim 28, characterised in that the first component contains at least one compound according to claim 9.

30. Pharmaceutical composition according claim 28, characterised in that the pharmaceutical compound of the second component is metabolised by the enzymes cytochrome P450 1A2, 3A4, 2C9 or 2C19.

31. A method of improving the bioavailability of a compound that is orally administered to a person comprising administering a ginger fraction according to claim 8 along with the compound to the person, wherein the ginger fraction is administered in an effective amount to inhibit cytochrome P450 (CYP) enzymes from metabolizing the compound.
